(19) United States
(12) Patent Application Publication
(10) Pub. No.: US 2017/0065261 A1
(43) Pub. Date: Mar. 9, 2017

(54) SPECIMEN COLLECTION DEVICE

(71) Applicant: EVE MEDICAL INC., Toronto (CA)

(72) Inventors: Victoria Jessica Ching, Toronto (CA); Evan Moses, Toronto (CA)

(21) Appl. No.: 15/309,098

(22) PCT Filed: May 5, 2015
(86) PCT No.: PCT/CA2015/050468

§ 371 Date: Nov. 4, 2016

Related U.S. Application Data
(60) Provisional application No. 61/988,657, filed on May 5, 2014.

(51) Int. Cl. A61B 10/02; A61B 10/00

(52) U.S. Cl. CPC A61B 10/02; A61B 10/0291; A61B 10/0045; A61B 2010/0216; A61B 2010/0074

(57) ABSTRACT

Various example specimen collecting devices are disclosed for the collection of biological samples. The specimen collection device is comprised of an elongate shaft and a distal sampling member, where the distal sampling member includes a plurality of bristles for collecting and sampling a biological fluid. Various example embodiments are provided in which the bristles of the distal sampling member are spatially configured and oriented for collection and elution of biological fluids that may contain cells, such as vaginal fluids.

### CROSS-REFERENCE TO RELATED APPLICATION

This application claims priority to U.S. Provisional Application No. 61/988,657, titled “SPECIMEN COLLECTION DEVICE” and filed on May 5, 2014, the entire contents of which is incorporated herein by reference.

### BACKGROUND

The present disclosure relates to an apparatus for obtaining a biological specimen from a body orifice and can be employed to collect samples such as biological tissues, cells and/or fluids.

Typically, specimens collected from body cavities are done so by means of a swab, which is generally comprised of a handle or shaft and a wound-cotton tip which is used for absorbing fluid or semifluid samples. These samples are often eluted into a media for the purpose of sample preservation or extraction. In other cases, a brush or scraper is used to scrape cellular material from a cellular wall or surface. Similarly, such cells are often stored or processed for analysis or diagnosis using a liquid medium.

There are a number of shortcomings which hamper the performance of a cotton-tipped swab for this use. To work effectively, wound cotton must absorb a sample of specimen, and this is done in uneven concentrations across the swab tip, with more material being concentrated at the surface of the swab's volume. More significantly, cotton swabs often exhibit difficulty in releasing or eluting fluids or biological materials into a medium. In such case, important biological material may be left inside the swab and therefore wasted. Furthermore, wound swabs require a separate and distinct manufacturing process.

Attempts have been made to improve upon the performance of the cotton swab by changing the wound materials, such as with the Dacron swab, or by using flocking to align fibers around the swab tip. In the former case, the problems with variable absorbency and elution of the sample are retained, in the latter, flocking requires a complex and advanced manufacturing process. Furthermore, these specimen collection devices work best with specimens that are liquid, mucus, or semifluid in composition, and are not optimal for scraping of cellular material from cellular or epithelial walls, or other surfaces.

Brushes and other scraping devices have been used to improve the performance of these specimen collection devices, particularly in the area of vaginal sampling for the purposes of cytology. While these specimen collection devices are adept at scraping cellular material, they are often unable to retain fluids which may contain important biological information useful for the purposes of analysis, diagnosis or research.

### SUMMARY

Various example specimen collecting devices are disclosed for the collection of biological samples. The specimen collection device is comprised of an elongate shaft and a distal sampling member, where the distal sampling member includes a plurality of bristles for collecting and sampling a biological fluid. Various example embodiments are provided in which the bristles of the distal sampling member are spatially configured and oriented for collection and elution of biological fluids that may contain cells, such as vaginal fluids.

Accordingly, in one aspect, there is provided a sample collection device comprising:
an elongate shaft having a longitudinal axis associated therewith; and
a flexible distal sampling member connected to a distal portion of said elongate shaft, said flexible distal sampling member comprising:
   a flexible planar elongate bristle support member extending along the longitudinal axis; and
   a plurality of bristles extending from opposing sides of said flexible planar elongate bristle support member;
wherein said flexible planar elongate bristle support member has cross-sectional dimensions, within a plane perpendicular to the longitudinal direction, such that a ratio of thickness to width lies between 1:3 and 1:9, and such that said flexible planar elongate bristle support member permits flexion in a first transverse direction associated with its thickness while resisting flexion in a second transverse direction associated with its width.

In another aspect, there is provided a sample collection device for collection of a vaginal sample during insertion into a vaginal cavity, comprising:
an elongate shaft having a longitudinal axis associated therewith; and
a flexible distal sampling member connected to a distal portion of said elongate shaft, said flexible distal sampling member comprising:
   a planar elongate bristle support member extending along the longitudinal axis; and
   a plurality of bristles extending from at least one side of said planar elongate bristle support member, and wherein said bristles are spatially arranged for retention of cellular material and biological liquid therebetween when said flexible distal sampling member is contacted with, and moved relative to, a tissue surface within the vaginal cavity; and
wherein an average spatial separation between adjacent bristles is between approximately 0.35 mm and 0.65 mm.

In another aspect, there is provided a sample collection device comprising:
an elongate shaft having a longitudinal axis associated therewith;
a distal sampling member connected to a distal portion of said elongate shaft, said distal sampling member comprising:
   a planar elongate bristle support member extending along the longitudinal axis; and
   a plurality of bristles extending in perpendicular directions from at least one side of said planar elongate bristle support member;
wherein said bristles have an elongate cross-sectional shape characterized by an elongate axis; and
wherein said bristles are angled relative to the longitudinal axis, such that an oblique angle is formed between the elongate axis and the longitudinal axis.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 is a perspective view of an example specimen collection device with the specimen collection portion detached from the elongate shaft.

FIG. 2A is an elevation view of an example configuration of the elongate shaft of a specimen collection device.

FIG. 2B is an elevation view of an example configuration of the elongate shaft of a specimen collection device.

FIG. 2C is an elevation view of an example configuration of the elongate shaft of a specimen collection device.

FIG. 3A is an elevation view of an example specimen collection device with an integral specimen collection portion.

FIG. 3B is an elevation and cross-section view of an example specimen collection device with the specimen collection portion detached from the elongate shaft.

FIG. 3C is an elevation and cross-section view of an example specimen collection device with the detachable specimen collection portion connected to the elongate shaft.

FIG. 3D is an elevation and cross-section view of an example configuration of the elongate shaft of a specimen collection device where it connects to the specimen collection portion.

FIGS. 4A and 4B shows cross-section and perspective view of several example connection mechanisms between the elongate shaft and specimen collection portion of a specimen collection device.

FIG. 4C is a cross-section view of an example connection between the elongate shaft and specimen collection portion of a specimen collection device.

FIG. 5A is a perspective view of an example specimen collection portion of a specimen collection device.

FIGS. 5B-I show top views of an example configurations of bristles upon the collection head of a specimen collection device.

FIGS. 6A-C illustrate an example implementation of a sample collection device in which the bristles are oriented such that they remain perpendicular to a helical direction of travel during simultaneous extension and rotation of the elongate shaft.

FIGS. 7A-E provide perspective views of the specimen collection portion of various example specimen collection devices.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately”, whether or not they are used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure. Unless otherwise specified, the phrase “approximately”, as used herein, refers to a range of plus or minus 25%.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

The term “brush” as used herein is used to denote a sampling component that is connected or connectable to a stem or handle, usually elongate, comprising a clump, bunch or group of bristles, hair or other similar flexible, semi-flexible, or rigid elongate strands, laminar flaps or the like. Whilst reference is made throughout the present specification to the collection of a sample within the bristles of a brush or brush-like device, it is to be understood that the reference to “bristles” is used to include the hairs or other similar flexible, semi-flexible or rigid elongate strands, laminar flaps or the like of a brush or brush-like device.

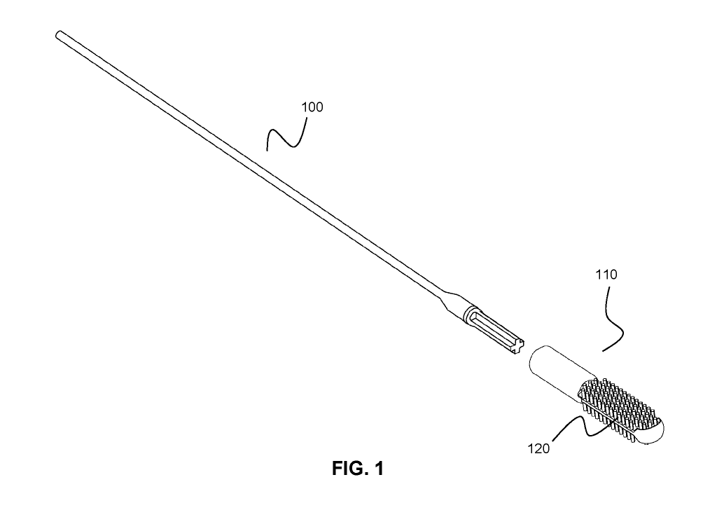

Embodiments of the present disclosure provide a sample collection device and associated method which may be suitable for collecting both cellular material and fluids, of retaining these materials with minimal loss, and of successfully eluting or releasing these materials when needed. FIG. 1 illustrates an example specimen collection device according to an embodiment of the present disclosure. The sample collection device has an elongate shaft 100 and a distal sampling member (a sample collection portion) 110. Distal sampling member 110, which includes a plurality of bristles 120, may be integrally formed with elongate shaft 100, or separated formed, and connectable, to elongate shaft 100. The distal sampling member extends along a longitudinal axis of the elongate shaft.

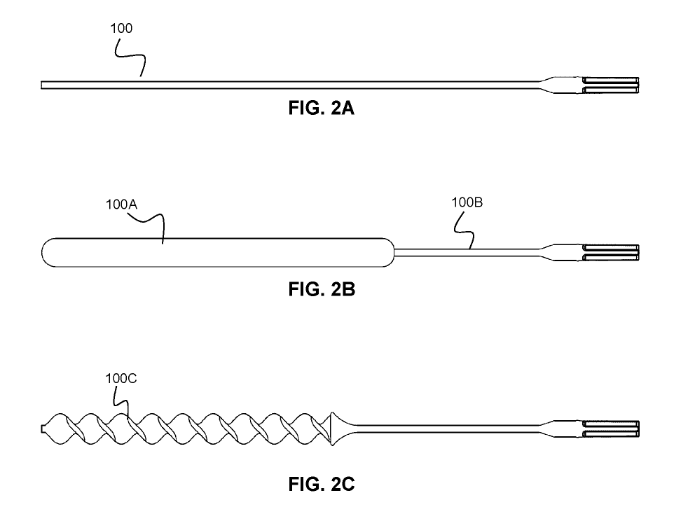

The elongate shaft 100 is connected to the distal sampling member 110 and functions to enable a user to hold the sample collection device and to direct the distal sampling member 110 to a collection site. In one embodiment, it may be made from plastic or such other material with sufficient elasticity so that it can be bent or otherwise deformed without permanent alteration of its shape or functionality. In this or other embodiments, the dimensional properties of the elongate shaft 100 may be such that its cross-sectional diameter may be relatively small in comparison to its length, such that the elongate shaft is flexible due to its dimensional properties in place of, or in addition to, its material properties, as shown, for example, in FIG. 2A. In any of these embodiments, the elongate shaft 100 may be formed from a material known to tolerate both prolonged arrangement in a curved position and heat treatment without permanent deformation. Furthermore, the material, dimensional, or other physical properties of the elongate shaft 100 may be selected to be sufficiently elastic to preclude breakage in normal operation of the device.

Example devices according to the present disclosure may be manufactured, for example, using standard processes such as injection molding, and be comprised of such plastics as would maintain the desired characteristics of strength and flexibility, including but not limited to: nylon, high density polyethylene, low density polyethylene, polypropylene, high impact polystyrene, or polyolefin elastomer. In some example embodiments, bristles 120 are integrally formed with the remainder of the distal sampling member 110, such that the distal sampling member can be formed as a single integral component. Devices may be manufactured with several materials, or overmoulded, to produce parts of varying characteristics, or manufactured with other techniques such as may be envisioned by one skilled in the art. In some embodiments, the distal sampling member may be configured such that it can be formed from injection molding using a two-part mold having a single draw direction.

In one example embodiment, the cross-sectional dimension of the elongate shaft may vary along its length, as shown, for example, in FIG. 2B (where elongate shaft is formed in sections 100A and 100B having different diameters), so that the elongate shaft may be easily held by a user's hand or other mechanism for holding the elongate shaft. In yet another embodiment, the elongate shaft may be configured in order to interact with the user's hand, a mechanism, or an adjacent device or physical configuration in a specific way, such as in FIG. 2C (which includes a threaded portion 100C that is configured to cooperate with an extension mechanism to simultaneously longitudinally extend and rotate the shaft). Other configurations or embodiments of the elongate shaft are possible, and the illustrated examples are not intended to exclude embodiments such as these from the present disclosure.

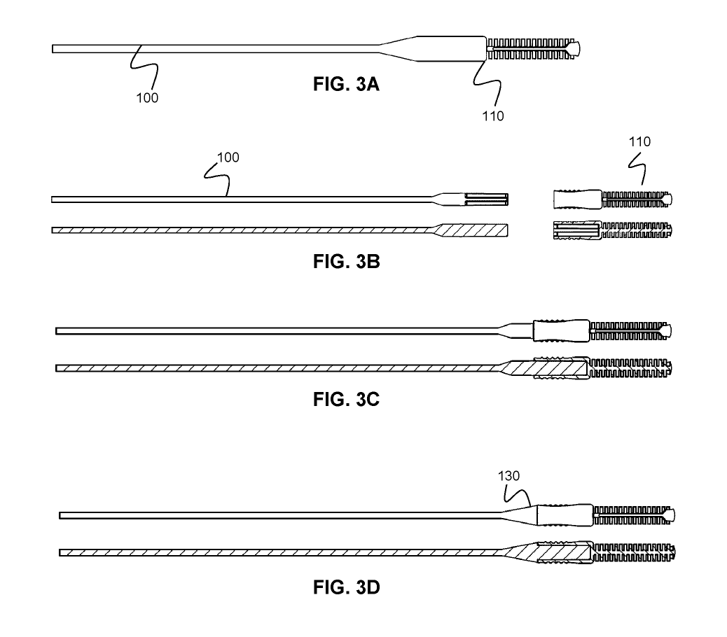

In one embodiment of the device, the elongate shaft 110 and distal sampling member 110 of the device are contiguous and integral, as shown, for example, in FIG. 3A. In such a configuration, the diameter of elongate shaft may be smaller at a specific breakpoint (not shown), thus allowing the elongate shaft to be broken in a controlled manner when a shear force is applied, such that the swab tip, and where desired other parts of the elongate shaft, can be broken off and separated from the elongate shaft 100 and/or device.

In an example embodiment of the device, the distal sampling member 110 may be detached from the elongate shaft, as illustrated in FIG. 3B, for storage, transport, processing, analysis or other use of a collected specimen. In this example embodiment the terminus (distal region) of the elongate shaft enables a secure connection between the elongate shaft and the distal sampling member 110, as shown in FIG. 3C. In one example embodiment, the terminal end of the elongate shaft may be smaller in diameter than the distal sampling member 110, allowing the users to differentiate between the two components and provide purchase so that the user may remove the distal sampling member 110. Conversely, the terminal end of the elongate shaft may be larger in diameter than the distal sampling member 110 to facilitate the separation of said components. In another example embodiment, the part of the elongate shaft leading up to the terminal end 130 may be flanged, so that the connection between the elongate shaft and the distal sampling member 110 appears to form a continuous surface, as shown in FIG. 3D.

In yet another example embodiment, the material or colour of the elongate shaft and distal sampling member may be different, so that the user may distinguish between the two and to facilitate the removal of the distal sampling member. This function may be performed by another means, through differing textures or finishes—for example, matte vs. gloss—applied to the two parts, or by visual indicators of the connection such as a diagram, arrows, or a circumferential line.

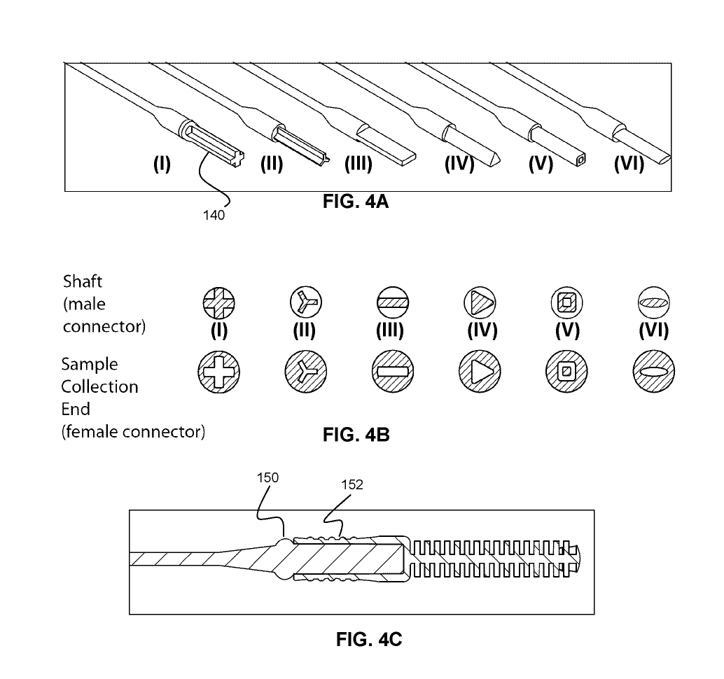

In example embodiments in which the collection portion of the device is detachable, the terminus of the elongate shaft end of the device and of the internal cavity have interlocking geometries that enable a secure fit between the two components, preventing accidental detachment of the collection portion from the elongate shaft, while nevertheless allowing the collection portion to be easily removed when required by a user. Conversely, the terminus of the elongate shaft could have an internal cavity which receives the male geometry of the distal sampling member 110 to secure a fit in a similar manner. In one example embodiment the terminus of the elongate shaft may have a cross section of two intersecting perpendicular geometries 140, as shown in FIGS. 4A (I) and 4B (I), forming a male insertion geometry in the shape of an extruded cross, while the distal sampling member may have the corresponding female geometry. Other geometries which allow a secure fit between elongate shaft and distal sampling member 110 may include, but are in no way limited to, those illustrated in FIGS. 4A and 4B, (II-VI).

Additionally, the geometry of either the male insertion geometry, or of the equivalent female element of the collection portion may have a ridge, bump or stop 150, which may allow the two components to be better secured together, or may act as a guide to facilitate proper positioning or alignment of the two components, as shown in FIG. 4C. The two components may have other complementary geometries which enable a secure or interlocking fit, such as a thread or screw, or another interlocking mechanism, such as a ball and socket, a latch, or a one-way valve. Furthermore, the connection between the two components may be such that it will resist disengagement under some threshold of rotational force, such that the torque created through rotation of the elongate shaft during the process of collecting a sample would not dislodge the mechanism, or allow the distal sampling member to be accidentally detached from the elongate shaft or vice versa. The connection may be such that only a torque force above the designed threshold, or in the opposite rotational direction, would allow the elongate shaft and distal sampling members to be detached from each other.

In various example embodiments of the present disclosure, the distal sampling member is configured to collect a biological specimen and may be employed to collect samples such as biological tissues, cells and/or fluids. The distal sampling member may include a connection end and a collection head.

The connection end may be comprised of a cylindrical outer section enclosing an interior void, the void optionally comprised of a female insertion geometry as described above, and may act a component that permits the collection portion of the sample collection device to be detached without handling the area of the distal sampling member on which a sample could be collected. In one embodiment, the connection may be imprinted with depressions, ridges or grips 152 (as can be seen in FIG. 4C) to enable a user to better grasp the connection end, and to remove it from the collection portion from the elongate shaft as desired.

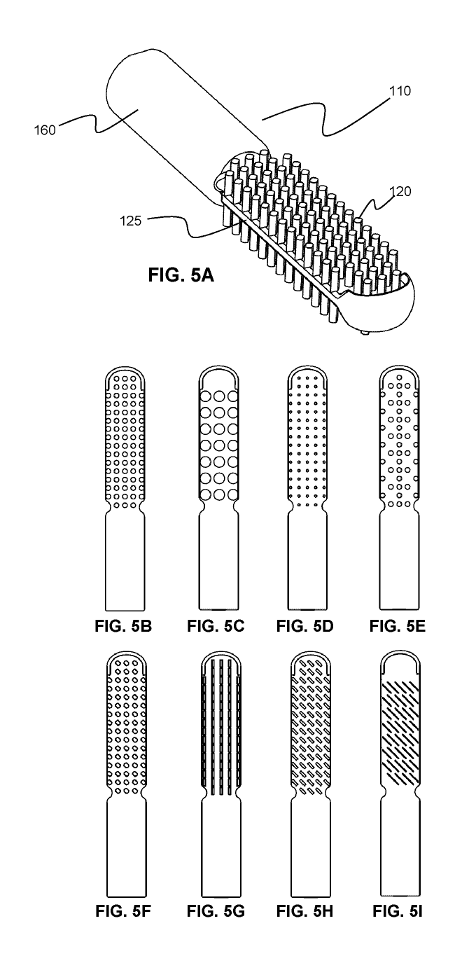

In one embodiment the collection head is designed as a brush suitable for collection of biological specimens, an example embodiment of which can be seen in FIG. 5A. The distal sampling member 110 may have two distinct components: a first (e.g. cylindrical) component 160 that connects to the elongate shaft, and a brush component composed of bristles 120 extending (e.g. perpendicularly) from a planar elongate bristle support member 125 (e.g. a central spine or blade). While most embodiments disclosed herein shown the planar elongate bristle support member as having a surface that is fully planar, it will be understood that in other embodiments, only a portion of the surface of the planar elongate bristle support member need form or approximate a planar surface.

In some embodiments, the brush bristles may be arrayed (periodically or randomly) on the surface of the planar elongate bristle support member so that the spaces between them can hold biological material and that the viscosity or surface tension of the material will allow it to be collected, retained and eluted by the brush in an effective manner. The bristles 120 may be arranged in a linear array, rectangular array, a hexagonal array, a circular array, a spiral array or another arrangement of bristles suitable to the collection, retention and elution of a specimen. For example, the bristles may be arranged in an offset grid such that any three adjacent bristles form an equilateral triangle

In one example implementation, shown in FIG. 5B, the bristles may have an average approximate cross-sectional width of 0.45 mm to 0.75 mm. The bristles may be spatially arranged in a manner to provide average spaces between adjacent bristles between approximately 0.35 mm to 0.65 mm, such that their configuration is suitable for the collection, retention, and elution of vaginal fluid. The cross-section of the bristles may be shaped such that a minimum cross-sectional dimension lies between 0.3 mm and 0.8 mm.

In other embodiments, the dimensions of the bristles or of the spaces between them may be bigger or smaller (illustrative examples are shown in FIGS. 5C and 5D), such that they are suitable for fluids or semifluids of different viscosities. For example, in an example embodiment for sampling less viscous fluids, such as saliva or blood, smaller average spaces may be provided between bristles, such as, for example, approximately 0.2 mm to 0.5 mm, whereas more viscous fluids, such as infected nasal mucous, may be suitably sampled with bristles that are an average of approximately 0.6 mm to 0.9 mm apart.

In yet another embodiment, the dimensions of the spaces between bristles may be varied (an illustrative example is shown in FIG. 5E) so that a single distal sampling member 110 would be suitable for collection of multiple fluids of differing viscosities, or for a single fluid with a range of potential viscosities. Similarly, a single brush head may have bristles of different diameters (not shown), and yet another embodiment is possible in which both the bristle diameter and spaces between bristles is varied on a single brush head.

In one embodiment of the device, the collection portion may be composed partially or wholly of an absorbent material, such that the process of collecting storing and releasing a sample is accomplished both by the material property of the collection portion as well as by the geometries and methods described herein.

In one example embodiment of the device, manipulation of the brush head, whether by flexion or compression, or by interaction with another mechanism or device, may effectively enlarge the spaces between bristles, which would further enable elution of the collected sample. Similarly, the device as a whole, or just the collection portion, may interact with another device, such as an agitation or sonication device, to improve elution or release of the sample from the collection portion of the device.

In one embodiment the bristles may be cylindrical in shape, as shown in FIG. 5B, or near-cylindrical, as shown in FIG. 5F.

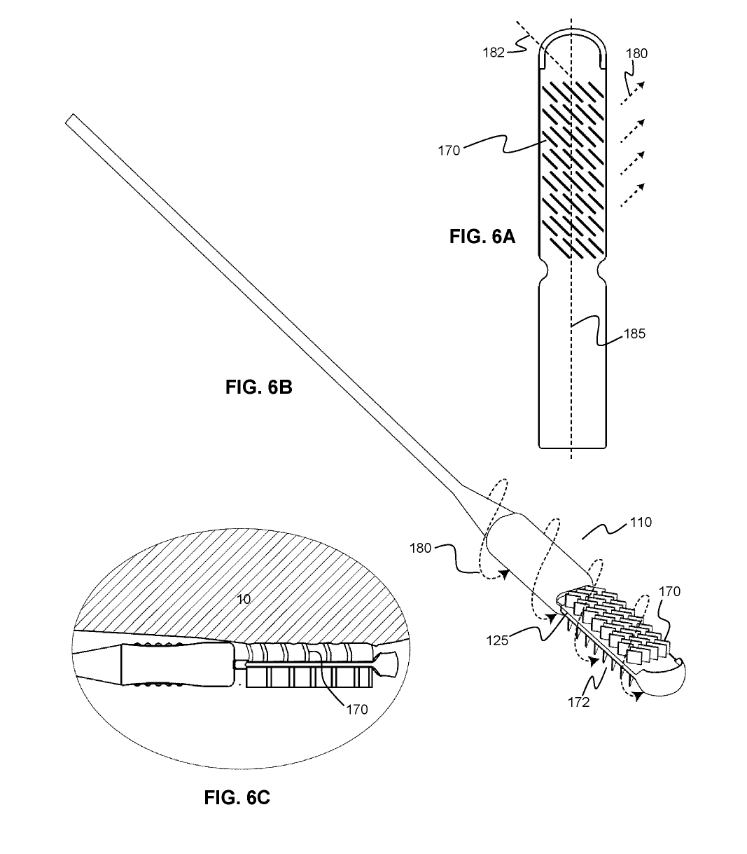

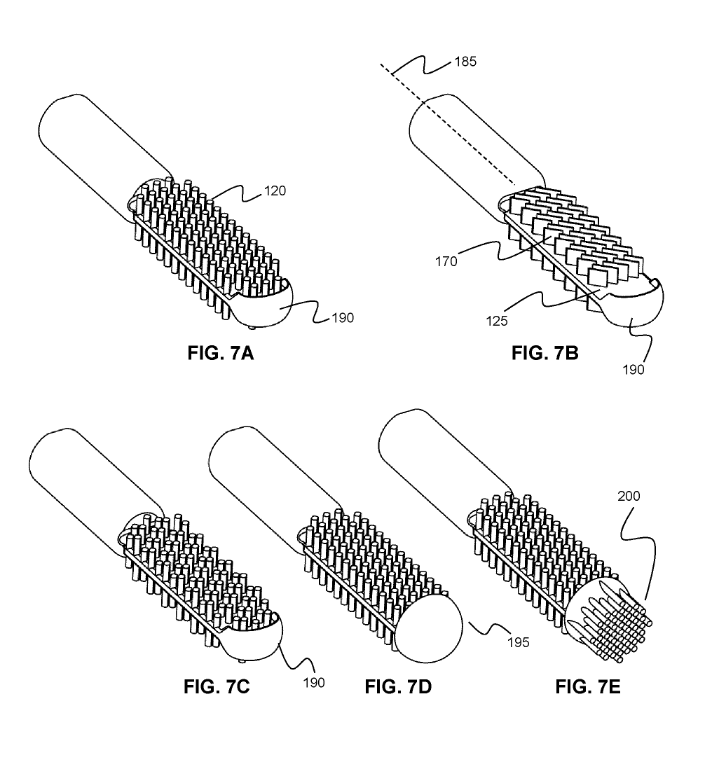

In another embodiment the bristles may be flat (e.g. planar), rectangular or finial in shape, and may be arrayed in any uniform orientation, examples of which can be seen in FIGS. 5G and 5H. The cross-sectional length of the bristle may be of differing dimensions relative to its width, and the array of bristles adjusted so that the bristles form an overlapping grid, as seen in FIG. 5I. The bristles may have a rectangular shape, as shown, for example, in FIGS. 5G-I, and in FIGS. 6A and 7B. In another example embodiment, the bristles may have an elliptical shape.

In one example embodiment, a length of said bristles less than or equal to one half of a width of said flexible planar elongate bristle support member.

In another example implementation, individual bristles may be arrayed with differing orientations on the same brush head.

In another embodiment, there may be bristles of differing cross-sectional dimensions and proportions of length to width on the same brush head.

In some embodiments, the flexible planar elongate bristle support member has cross-sectional dimensions, within a plane perpendicular to the a longitudinal axis of the shaft, such that a ratio of thickness to width lies between 1:3 and 1:9, and such that said flexible planar elongate bristle support member permits flexion in a first transverse direction associated with its thickness while resisting flexion in a second transverse direction associated with its width.

As shown in FIGS. 5H, 5I and 6A, the bristles may be arranged in a grid, wherein the bristles have an elongate cross-sectional shape characterized by an elongate axis. The elongate axis of each bristle may be angled at an oblique angle relative to axes of the grid. In other words, the bristles may have an elongate cross-sectional shape characterized by an elongate axis, where the elongate axis of each bristle is angled at an oblique angle relative to the longitudinal axis of the device. The oblique angle may lie between 45 degrees and 75 degrees.

Each bristle may have an elongate cross-sectional shape characterized by an elongate axis and a short axis, where a width of each bristle along the elongate axis is at least three times a thickness of each bristle along the short axis.

In some embodiments, the bristles may have length selected such that a maximum radial distance of said flexible planar elongate bristle support member, relative to the longitudinal axis, is greater than a maximum radial extent of said bristles, relative to the longitudinal axis.

In one example embodiment, the bristles may be oriented at an angle, such that when a torque is applied to the bristles during use (e.g. by a tissue surface), the bristles maintain an approximately perpendicular orientation relative to the tissue surface.

As noted above, in some embodiments, the bristles may extend from one or both sides of the planar elongate bristle support member, and may have an elongate cross-sectional shape characterized by an elongate axis, where the bristles are angled relative to the longitudinal axis, such that an oblique angle is formed between the elongate axis 182 and the longitudinal axis 185. An example of such an embodiment is illustrated in FIGS. 6A-6C.

This enables such a device to maintain an orientation of the bristles relative to a helical path 180 of the bristles, as the shaft is rotated and extended in a continuous motion, as shown in FIG. 6B. For example, the larger dimension of each bristle may be maintained in an orientation facing the direction of travel during extension and rotation, and the brush is able to collect more biological material.

In some embodiments, a housing may be configured to receive at least a proximal portion of the elongate shaft. The housing may incorporate an extension mechanism configured to extend and rotate the elongate shaft from said housing in the longitudinal axis, such that distal ends of said bristles trace a helical path during extension of the elongate shaft along the longitudinal axis. The helix angle characterizing the helical path may be selected to be between 20 degrees and 30 degrees. Examples of mechanism for producing helical motion (rotation and extension) of the shaft are described in PCT Patent Application No. PCT/CA2013/000991, titled “SPECIMEN COLLECTION DEVICE AND KIT”, filed on Nov. 27, 2013, which is incorporated herein by reference in its entirety. For example, in one example embodiment of the device, the shaft may have a helical segment which may rest within threading mechanism when the shaft is either in the retracted or extended position. In another embodiment, the shaft may be pushed into or out of a position in which it can engage threading mechanism during the operation of the device. According to one example embodiment, upon rotation of the shaft, a helical segment may interacts with a threading mechanism such that the rotation of the shaft is translated into lateral movement and the shaft is pulled through the mechanism along the shaft's length as it rotates.

In some embodiments, the bristles may be oriented such that during use, the elongate axis of the bristle is approximately perpendicular to a tangential line associated with the local motion of the end of the bristle. It will be understood that brush may be translated, rotated, or translated and rotated, during use.

In embodiments in which the bristles are oriented based on a pre-selected rotational direction of the elongate shaft, the elongate shaft may include one or more markings or features configured to indicated the correct rotational direction for use.

As shown in FIG. 6B, in one example embodiment, the bristles may extend from both opposing sides of the planar elongate bristle support member. The bristles on the opposing sides 172 may be oriented such that the orientation of the bristles on one side of said planar elongate bristle support member is symmetric to the orientation of the bristles on the other side of the planar elongate bristle support member under a rotation of 180 degrees about the longitudinal axis.

In one example embodiment of the disclosure, the bristles 120 are of uniform height, as shown in FIG. 7A. In another embodiment, the bristles are of varying lengths or shapes. In another example embodiment, the central spine or blade may be comprised of a surface which is not planar, allowing for differing orientations of bristles, or the bristles may protrude radially from the spine in a circular geometry around all or part of its circumference.

In yet another embodiment the bristles are joined at an angle with respect to the planar elongate bristle support member 125, as shown, for example, in FIG. 7B in which the planar bristles 170 are shown angled relative the longitudinal axis 185 of device, as described above.

FIGS. 7B and 7C show example embodiments in which the tip of the distal sampling member 110 is covered by a semi-circular partition 190 which reduce discomfort upon insertion of the device into a bodily orifice, and may protect the bristles from prematurely collecting a sample when inserted, and may also scrape additional cell material and deposit it on the brush head when the device is retracted from an orifice.

In another example embodiment the disclosure, the tip of the brush is rounded to form a semi-spherical cap 195 to minimize discomfort upon insertion into a bodily orifice, as shown, for example, in FIG. 7D. In yet another embodiment, the end or tip of the brush may be covered in further bristles 200 to maximize collection of material (FIG. 7E). These bristles may be configured longitudinally, or radially, or in some other configuration or combination of configurations such that they are different from the bristles emanating from the central spine. The bristles on the tip of the collection portion may be suitable for collecting a different type of sample or a sample from a different region than the other bristles of the collection head, such as from the cervical or rather than the vaginal walls, or from the pharynx rather than the palate or floor of the mouth. In another embodiment, the tip of the collection portion may be detachable from the remainder of the collection portion, so that the two different specimens, or specimens that are collected from two different areas, could be stored or analyzed separately.

In one embodiment, the bristle may be made from a flexible material such as low density polyethylene or polyolefin elastomer (POE) or other such suitable material, such that it may bend or flex with relative ease when pushed against a surface such as the skin or orifice of a user. In one embodiment, the bristle may be between 2 mm and 3 mm in length and with a cross-sectional dimension of between 0.35 mm and 0.65 mm. The flexion of the individual bristles is such that a user would not find the brush uncomfortable when pushed with significant force (e.g. greater than 20N) against their skin. Each bristle may flex independently so that when the brush passes over an uneven surface, individual bristles can maintain contact.

In one embodiment, the bristles may be composed of dimensions and materials such that they are sufficiently flexible to maintain contact with the sampling surface along their elongate cross-section, even in such cases where the surface may be uneven. In such case, the bristles should still be sufficiently rigid that they can resist buckling, even in the advent of a force of up to 10N applied at their tip. In one embodiment, such properties would be enabled by a bristle comprised of a low density polyethylene or polyolefin elastomer (POE) or other such suitable material, and be of length between 2 mm and 4 mm with an elongate cross section between 1.5 mm and 3.5 mm and a transverse cross-section between 0.1 mm and 0.5 mm. Equivalently, other dimensions with approximately this ratio of length and cross-sectional dimensions may be suitable and resist an equivalently scaled greater or lesser force as needed.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A sample collection device comprising:
an elongate shaft having a longitudinal axis associated therewith; and
a flexible distal sampling member connected to a distal portion of said elongate shaft, said flexible distal sampling member comprising:
a flexible planar elongate bristle support member extending along the longitudinal axis; and
a plurality of bristles extending from opposing sides of said flexible planar elongate bristle support member;
wherein said flexible planar elongate bristle support member has cross-sectional dimensions, within a plane perpendicular to the longitudinal direction, such that a ratio of thickness to width lies between 1:3 and 1:9, and such that said flexible planar elongate bristle support member permits flexion in a first transverse direction associated with its thickness while resisting flexion in a second transverse direction associated with its width.

2. The sample collection device according to claim 1 wherein an average spatial separation between adjacent bristles is between approximately 0.35 mm and 0.65 mm.

3. The sample collection device according to claim 1 wherein said bristles have a cross-section characterized by a minimum cross-sectional dimension between 0.3 mm and 0.8 mm.

4. The sample collection device according to claim 1 wherein said bristles are arranged in a rectangular grid.

5. The sample collection device according to claim 1 wherein said bristles are arranged in an offset grid such that any three adjacent bristles form an equilateral triangle.

6. The sample collection device according to claim 1 wherein said bristles have a rectangular shape.

7. The sample collection device according to claim 1 wherein said bristles are arranged in a grid, wherein said bristles have an elongate cross-sectional shape characterized by an elongate axis, and wherein the elongate axis of each bristle is angled at an oblique angle relative to axes of the grid.

8. The sample collection device according to claim 1 where each bristle has an elongate cross-sectional shape characterized by an elongate axis and a short axis, and wherein a width of each bristle along the elongate axis is at least three times a thickness of each bristle along the short axis.

9. The sample collection device according to claim 1 wherein said bristles have an elongate cross-sectional shape characterized by an elongate axis, and wherein the elongate axis of each bristle is angled at an oblique angle relative to the longitudinal axis.

10. The sample collection device according to claim 9 wherein the oblique angle is between 45 degrees and 75 degrees.

11. The sample collection device according to claim 1 wherein said bristles are have length selected such that a maximum radial distance of said flexible planar elongate bristle support member, relative to the longitudinal axis, is greater than a maximum radial extent of said bristles, relative to the longitudinal axis.

12. The sample collection device according to claim 1 wherein a length of said bristles less than or equal to one half of a width of said flexible planar elongate bristle support member.

13. The sample collection device according to claim 1 wherein said flexible planar elongate bristle support member and said bristles are integrally formed from a moldable material.

14. The sample collection device according to claim 1 wherein said bristles extend from said flexible planar elongate bristle support member in directions that are perpendicular to said flexible planar elongate bristle support member, such that said flexible distal sampling member is manufacturable using a two-part mold having a draw line perpendicular to said flexible planar elongate bristle support member during use.

15. A sample collection device for collection of a vaginal sample during insertion into a vaginal cavity, comprising:
an elongate shaft having a longitudinal axis associated therewith; and
a flexible distal sampling member connected to a distal portion of said elongate shaft, said flexible distal sampling member comprising:
a planar elongate bristle support member extending along the longitudinal axis; and
a plurality of bristles extending from at least one side of said planar elongate bristle support member, and wherein said bristles are spatially arranged for retention of cellular material and biological liquid therebetween when said flexible distal sampling member is contacted with, and moved relative to, a tissue surface within the vaginal cavity; and
wherein an average spatial separation between adjacent bristles is between approximately 0.35 mm and 0.65 mm.

16. The sample collection device according to claim 15 wherein said bristles have a cross-section characterized by a minimum cross-sectional dimension between 0.3 mm and 0.8 mm.

17. The sample collection device according to claim 15 wherein said bristles are arranged in a rectangular grid.

18. The sample collection device according to claim 15 wherein said bristles are arranged in an offset grid such that any three adjacent bristles form an equilateral triangle.

19. The sample collection device according to claim 15 wherein said bristles have a rectangular shape.

20-23. (canceled)

24. The sample collection device according to claim 15 wherein said bristles are have a length selected such that a maximum radial distance of said planar elongate bristle support member, relative to the longitudinal axis, is greater than a maximum radial extent of said bristles, relative to the longitudinal axis.

25. The sample collection device according to claim 15 wherein a length of said bristles is less than or equal to one half of a width of said planar elongate bristle support member.

26-36. (canceled)
